(19) United States
(12) Patent Application Publication
(10) Pub. No.: US 2024/0362673 A1
(43) Pub. Date: Oct. 31, 2024

(54) SYSTEMS AND METHODS FOR GOAL-DRIVEN CONTEXTUAL OMNICHANNEL OPTIMIZATION USING WELL-ROUNDED ARTIFICIAL INTELLIGENCE

(71) Applicant: Aktana, Inc., San Francisco, CA (US)

(72) Inventors: Pinchas BEN-OR, Issaquah, WA (US); Satya DHANUSHKODI, Cupertino, CA (US); Anwar SHAIKH, San Francisco, CA (US); Simon ROBINS, Haifa (IL); Vadim KAGANSKIY, San Francisco, CA (US); Diana SCHNEIDER, Modin (IL); Debabrata PATI, Pune (IN); Michael STEINER, San Francisco, CA (US); Nitin MALI, San Mateo, CA (US); Igor PUTRENKO, Fremont, CA (US); Vikram KAMTHE, Pune (IN); Owen BIDDER, Oakland, CA (US); Craig VOLONOSKI, San Carlos, CA (US); Marc COHEN, San Francisco, CA (US); Derek CHOY, San Francisco, CA (US)

(21) Appl. No.: 18/655,651

(22) Filed: May 6, 2024

Related U.S. Application Data
(63) Continuation of application No. PCT/US22/50155, filed on Nov. 16, 2022.
(60) Provisional application No. 63/280,596, filed on Nov. 17, 2021.

(51) Int. Cl. G06Q 30/0242; G06Q 30/0202; G06Q 30/0204

(52) U.S. Cl. CPC G06Q 30/0244; G06Q 30/0202; G06Q 30/0204

(57) ABSTRACT

Systems and methods for using models to optimize suggestions for long-term and short-term goals are disclosed. In one aspect, a method may include receiving input data associated with promotion of a product to one or more target entities, and defining a plurality of temporal goals for attainment based at least in part on the input data. The plurality of temporal goals may be classified into at least two categories of different timescales. The method may further include providing the plurality of temporal goals and a set of constraints to an optimization model comprising an objective function, and using the optimization model to generate a set of multi-dimensional actions with predicted action values for at least a subset of the plurality of channels. The predicted action values can collectively maximize an impact value of the objective function while balancing a feasibility of the plurality of temporal goals with respect to one another.

### CROSS-REFERENCE

This application is a continuation of International Patent Application No. PCT/US22/50155, filed on Nov. 16, 2022, which claims priority to U.S. Provisional Patent Application No. 63/280,596, filed on Nov. 17, 2021, each of which is entirely incorporated herein by reference.

### BACKGROUND

When an enterprise is trying to retain as many satisfied customers as possible and have meaningful engagement with potential targets, long-term and short-term considerations or goals often need to be considered and balanced. Although artificial intelligence has been used to make predictions as to which decisions may impact sales and marketing of products or services, more sophisticated analysis may be needed to maximize sales and marketing goals (like sales or activity targets) and improve customer interactions/experience over time. In the healthcare space, pharmaceutical sales representatives (pharma reps) may use many different channels to communicate with health care providers (HCPs). For a pharmaceutical company, quantitatively analyzing best practices in light of variations may be challenging, particularly when there is a need to consider and balance across multiple products, channels, reps, HCPs, and customers.

### SUMMARY

There is a need for systems and methods that can utilize results from different machine-learned predictions and user-defined strategies to maximize/achieve at least one long term goal and a plurality of short term goals that enhance communication effectiveness and engagement between one or more actor types (e.g. pharma reps) and one or more target entities (e.g. HCPs), across a multitude of channels over one or more time periods. The present disclosure can address at least the above need, by using machine learning models with rules and constraints to provide suggested actions to facilitate achieving of both long-term and short-term goals.

In an aspect of the disclosure, a method comprises receiving input data associated with promotion of a product to one or more target entities: defining a plurality of temporal goals for attainment based at least in part on the input data, wherein the plurality of temporal goals are classified into at least two categories of different timescales: providing the plurality of temporal goals and a set of constraints to an optimization model comprising an objective function, wherein the set of constraints comprise action-type constraints, channel constraints, channel capacity constraints, pacing constraints, and/or channel fatigue constraints across a plurality of channels, wherein the plurality of channels are used to enable or facilitate communications between one or more actor types and the one or more target entities; and using the optimization model to generate a set of multi-dimensional actions with predicted action values for at least a subset of the plurality of channels, wherein the predicted action values collectively maximize an impact value of the objective function while balancing a feasibility of the plurality of temporal goals with respect to one another.

In another aspect of the disclosure, an artificial intelligence (AI)-driven omnichannel management system comprises: an aggregation module that is configured to combine various types of data from across a plurality of channels including push channels and pull channels: a standardization module that is configured to convert the combined data into a set of standardized data using a common data model/framework; and an action candidate generation module that is configured to (1) use the standardized data from the common data model/framework to generate a first set of action candidates to achieve a plurality of predefined short-term sales and marketing goals, and (2) generate a second set of action candidates to achieve a plurality of predefined long term goals; and an optimization module that is configured to process a set of fully formed action candidates, and generate one or more recommendations for optimizing or enhancing interactions by one or more actor types with one or more target entities in association with promotion of a product to the one or more target entities.

In a further aspect of the disclosure, a method for simulating a behavior of a system over a plurality of time periods comprises: (i) obtaining datasets from a plurality of data stores: (ii) combining the datasets with a suggestion record to generate a set of model features: (iii) generating a model output probability using a plurality of machine learning (ML) models based at least on the set of model features; and (iv) converting the model output probability to behavioral indicators using a rule-based model with segmentation information, to simulate a behavior of the system for a first time period, wherein the simulated behavior of the system for the first time period is used as input for simulating a future behavior of the system for a second time period that is subsequent to the first time period.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A illustrates a system for optimizing sales effectiveness and communication effectiveness between one or more actor types, and one or more target entities:

FIG. 1B illustrates an example of the architecture in FIG. 1A according to some embodiments:

FIG. 2 illustrates examples of inputs that may be provided to the system for optimization;

FIG. 3 illustrates an example of email timing optimization according to some embodiments:

FIG. 4 illustrates an example of the optimization of rep interactions with different HCPs:

FIG. 5A is a flowchart showing the manner in which data points, rules and segments are built upon, according to some embodiments:

FIG. 5B shows a goal metric tracking segment model according to some embodiments:

FIG. 6 is a schematic showing the types of inputs that are provided to the system for optimization;

FIG. 7 illustrates a system that is configured to interface with external models and analytic access points:

FIG. 8 shows the calculation for the expected action value for each fully formed suggestion candidate:

FIG. 9 illustrates a content targeting models (CTM) analytics architecture according to some embodiments:

FIGS. 10-1 and 10-2 illustrate a behavior simulator in accordance with some embodiments; and

FIG. 11 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure is directed to a versatile artificial intelligence (AI)-driven platform that can facilitate or enhance interactions and engagement between one or more actor types, and one or more target entities. The interactions may relate to sales, marketing, advertising or promotion of one or more products or services to one or more target entities. The platform is built as a well-rounded AI engine having several key elements that are combined in unique ways to solve real-world business problems. As an example, machine learning, optimization and algorithms can be collectively combined as described herein, to explain various actions and rules to stakeholders and customers in a transparent manner. The key elements of the well-rounded AI engine include (1) machine learning models, (2) rule/expert systems having rules and feature sets, (3) constrained optimization, (4) information solicitation and learning feedback loop. (5) natural language processing, and (6) contextual business intelligence and insights. The combination of the above elements will be apparent in light of the description of the examples herein.

In some embodiments, the one or more actor types may comprise one or more enumerated actors including sales representatives, district managers, or medical science liaisons that are associated with the promotion of the product to the one or more target entities. In other embodiments, the one or more actor types may comprise automated systems including marketing automation systems or web portal management systems.

In some embodiments, the target entities may comprise one or more healthcare providers (HCPs), healthcare organizations (HCOs), or healthcare institutional accounts. Examples of HCPs may include a doctor, nurse, nurse practitioner, physician assistant, technician, physical therapist, occupational therapist, health aide, respiratory therapist, or clinical psychologist.

In some embodiments, a product may be a health product. The product may be a pharmaceutical product. The product may comprise a drug, a therapeutic, a health treatment, a medical device, or any health or healthcare product to treat one or more health or disease-related conditions. A drug may be a prescription drug or an over-the-counter (OTC) drug. A health treatment may be a therapy or surgery.

The AI-driven platform can be configured to ensure that every customer experience is tailored to individual preferences and needs, thereby helping customers (for example, life science companies) to strengthen their relationships with target entities (for example, HCPs or HCOs) in achieving better or improved patient care.

The AI-driven platform is omnichannel, as in the AI-driven platform can orchestrate actions over a plurality of promotional/communication channels to optimize interactions and engagement between one or more actor types, and one or more target entities. The orchestration can include coordinating different actions across multiple channels in a synchronized fashion, to ensure that the experience for actor types as well as target entities is seamless, impactful and rewarding for parties on all sides.

Given there are often multiple available channels between actor types and target entities, and numerous variables/constraints involved, this presents an omnichannel challenge which can be classified as an optimization problem. The steps towards solving the omnichannel challenge include understanding the customer's needs, creating engaging customer experiences, and ensuring flawless execution, which can be accomplished using the systems and methods described herein. Although the majority of this disclosure is described in the context of interactions between life science companies, pharmaceutical sale representatives and healthcare providers, it should be noted that the present disclosure is not limited thereto, and may be applicable to a variety of other industries or fields involving customer experience and sales/marketing/promotion of products.

The AI-driven platform herein can integrate with a full range of data sources, analytics, and marketing technology. The platform includes a contextual intelligence engine (CIE) which is configured to optimize actions across different segments of target entities, across multiple channels, and across multiple temporal periods. The CIE may include one or more machine learning modules, customer journey management tools, and tools for execution management workflows as further described herein. The CIE may be referred to interchangeable herein as an optimization module, an optimization engine, an optimizer, and the like. In some instances, an optimization module, an optimization engine, or an optimizer may a component or subcomponent that is integrated into as part of the CIE. In other instances, the optimization module, the optimization engine, or the optimizer itself may be the CIE. In further instances, the CIE may comprise a plurality of optimization modules, optimization engines or optimizers. It should be appreciated by those skilled in the art that various configurations or modifications to the embodiments described herein may be implemented.

The CIE can be configured to process data, rules, campaigns and models from any source. The CIE may also utilize a common data model and framework for OC action evaluation, and recommendations/optimization incorporating feedback from multiple channels.

The CIE can also synthesize suggestions across multiple brands and products while incorporating practical considerations for each customer. For example, the CIE can be configured to combine/synthesize recommendations across multiple brands/products. The CIE platform described herein can be used for, or used by brand managers. For example, the CIE platform can be used to generate visual reports, notifications and recommendations to brand managers. Brand managers can use the CIE platform to create one or more campaigns for different brands/products that are targeted to the same or different segments of HCPs. The CIE platform can be used by brand managers to create/generate targeted content for sales and marketing. In some instances, the CIE platform can be used by brand managers for field resource allocation (which impacts cost allocation), for example for optimal deployment of teams of reps. The CIE platform can be used by brand managers to create campaign goals, monitor the performance of the campaign as it progresses, and to alter campaign strategy based on feedback provided from the CIE platform in order to achieve campaign goals.

The CIE can include granular controls that can enable precision optimization based on commercial priorities. The granular controls or levers can be adjusted to influence action values and monitor action values. Examples of those controls or levers may be based on one or more of the following: cost, value, impact factor, urgency, recency, channel propensity, proximity, rep engagement, time to engage, account priority, factor priority, etc.

As examples, account priority may relate to which accounts are of higher priority to target and engage with, versus lower priority accounts. Impact factor may refer to an impact of an action or interaction of a pharma rep with a HCP. Urgency, recency or time to engage may refer to a timing or frequency of interactions between a pharma rep and a HCP. A channel propensity may refer to a likelihood of a HCP or a rep preferring or utilizing one channel over another. Proximity may refer to a physical distance between a rep and a HCP. Factor priority may be associated with a score that is based on a value of an interaction between a pharma rep and a HCP.

The CIE is AI-driven, with modular optimization that balances expected impact, execution feasibility and capacity/resource spend. The CIE is configured to take into account and optimize asset utilization. For example, the CIE can recommend optimal utilization of assets (e.g., field reps, medical science liaisons-MSLs) for field channels when generating recommendations, based on a total demand for all available field resources across all candidate sales/marketing activities.

The CIE has an architecture that enables balanced, dynamic approach between machine learning (ML) and expert guidance for real-world efficacy. The CIE is configured to reconcile/optimize for both short-term campaign execution and long-term engagement strategies. The CIE may include an optimizer (objective function with constraints) that is configured to solve for expected OC action values while balancing a feasibility of long-term and short-term goals and the constraints. As an example, the CIE can be configured to consider medium-term engagement targets, pacing constraints, channel fatigue constraints across multiple brands and channels when generating recommendations, to maximize the objective function impact. The CIE can also include channel selection and content selection models that learns from associated long/short term goal metrics to continuously refine the models.

The CIE is built for omnichannel (OC), and can generate OC actions with context for delivery. It can enable cross-channel optimization across both push and pull marketing strategies, including unique customer-defined channels. For example, the CIE can be configured to optimize HCP interactions through both field and medical suggestions, as well as digital channel triggers, both push and pull, along with respective content. Content can include emails, brochures, topics, websites, social media, etc.

The system/platform can be built for experimentation facilitation, which enables the system to experiment, during production, in order to maximize and accelerate the ML models learning. As described herein, the system can be configured to estimate/calculate values, and to select an optimal option or suggestion given the constraints imposed. Experimentation involves intentionally experimenting with and recommending sub-optimal options or suggestions to an actor type (e.g. reps), collecting information from the responses, and feeding back to the ML models for updating and for the models to learn.

Risk appetite as used herein corresponds to experimentation appetite. A customer may express appetite as to how much risk they are willing to take in experimenting with suggestions that are sub-optimal. For example, a customer may wish to experiment on no more than 5% or 10% of the suggestions provided by the system, which can vary by segment and product. Accordingly, customers can use the system to control the degree or extent to which to experiment with sub-optimal suggestions. The results or learnings from the experimentation can be fed back to the system to further refine and optimize options or suggestions.

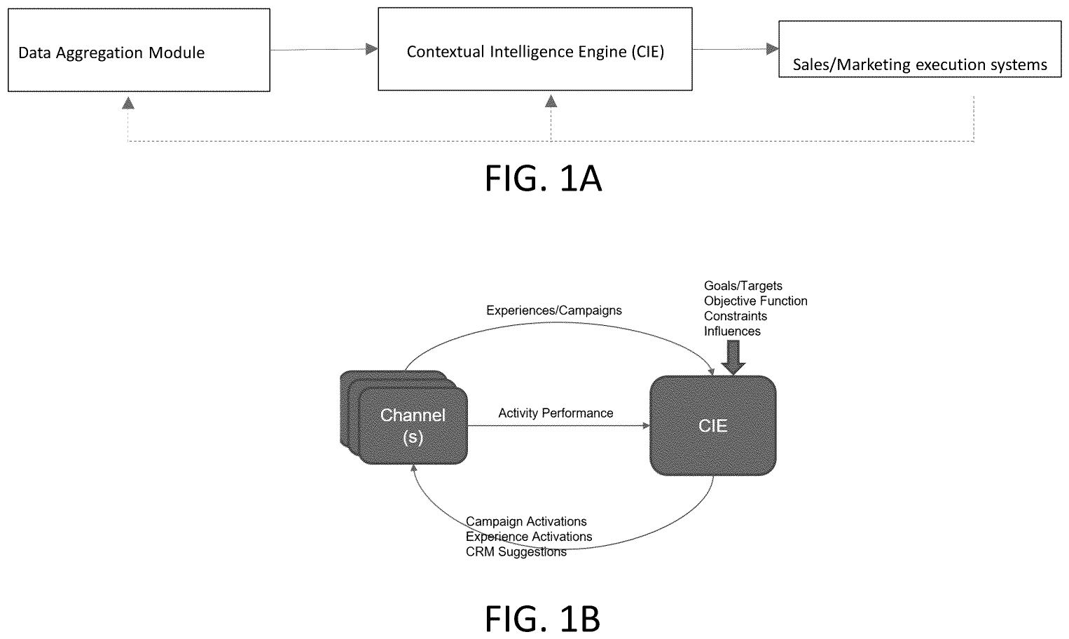

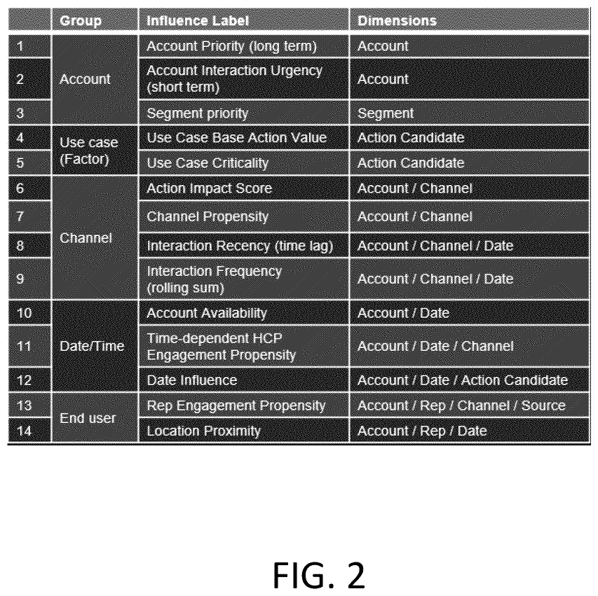

The CIE can offer scenario management to help evaluate and understand OC campaign/strategy trade-offs using an overall major goal, and sub goals (key performance indicators-KPIs). The CIE can also include experimentation/simulation capabilities as described with reference to the behavior simulator in FIGS. 10-1 and 10-2 later. The CIE can also include KPI tracking and reporting provides the confidence to continue or adjust configuration to best suit strategy and ever-changing market needs.

Disclosed herein are systems and methods for optimizing engagement effectiveness between one or more actor types (e.g. pharmaceutical sales reps—pharma reps) and one or more one or more target entities (e.g. HCPs), by balancing at least one long term goal for a customer (e.g. pharmaceutical company) with a plurality of short term goals relating to sales, marketing or promotion of a product or service that is being offered. The short term goals may be complementary to the long term goal, or may be sub-steps of the long term goal. In some instances, the short term goals may include actions to be taken by the actor types, responses elicited from the target entities, or both of the aforementioned. An example of a long term goal may include enhanced targeting of one or more HCPs, or interactions with those HCPs, towards achieving a more engaging experience with the HCPs. Examples of short term goals may include enabling a pharma rep to more appropriately react to HCP-led or HCP-initiated engagement, assisting with access challenges (e.g. ability of a HCP to access certain channels and/or content), or sharing of new clinical data with the HCPs, or coordinating rep-HCP interactions around an event (e.g. a conference) that is of potential interest to the HCP. To perform this optimization, the system may use an optimization module (e.g. CIE described elsewhere herein) that applies a value function to multiple inputs and applies constraints to the value function to obtain an optimized action set. An action may be a suggestion or recommendation to a rep to communicate with one or more HCPs over one or more channels, with specific content that is targeted and individualized for each HCP.

In some instances, a long term goal of a customer (e.g. pharma company) may include foundational targeting, such as prioritization of HCPs (or HCP segments) based on long term opportunities for engagement or sales to the associated HCOs. A long term goal may be updated as frequently as necessary, for example every 6-12 months by the customer (e.g. pharma company).

In some instances, short term goals or metrics may include (1) AI-based monitoring of sales data to identify any significant deviations, (2) automatic detection of new prescribers or newly diagnoses patients, based on recent drug product sales or medical claims: (3) strategic actions to progress actor types and/or target entities through configurable, complex journeys involving their interactions, or (4) pre-defined HCP engagement sequences that are designed to drive day-to-day re engagement with one or more HCPs. The short term goals or metrics may be updated on a shorter timescale than the long term goal. For example, the short term goals or metrics may be updated daily, weekly or monthly.

As described herein, the system can provide practical guidance to reps on which HCPs (or segments) to engage, when to engage, how to engage (i.e. which channels to utilize), what communications or content to provide to the HCPs, etc. in order to optimize engagement between the reps and HCPs. The guidance can be updated daily, based on full historical visibility of past interactions with the HCPs or other similar experiences/interactions. The guidance can also balance short term engagement needs with long term commercial opportunity.

FIG. 1A illustrates a system for optimizing sales effectiveness and communication effectiveness between one or more actor types, and one or more target entities. The system may include a data aggregation module, a contextual intelligence engine (CIE), and a customer relationship management (CRM) component.

The data aggregation module can be configured to receive input data associated with promotion of a product or service to one or more target entities. The aggregation module can be configured to combine various types of data from across a plurality of channels including push channels and pull channels. In some instances, a standardization module can be configured to convert the combined data into a set of standardized data using a common data model/framework.

A product may be a healthcare product. A product may be a pharmaceutical product. A product may comprise a drug, a therapeutic, a health treatment, a medical device, or any health or healthcare product to treat one or more health or disease-related conditions. A drug may be a prescription drug or an over-the-counter (OTC) drug. A health treatment may be a therapy or surgery.

The target entities may comprise one or more healthcare providers (HCPs), healthcare organizations (HCOs), or healthcare institutional accounts. Examples of HCPs may include a doctor, nurse, nurse practitioner, physician assistant, technician, physical therapist, occupational therapist, health aide, respiratory therapist, or clinical psychologist.

The input data may comprise market data, strategies, analytics, content, context, campaigns/rules, or models from one or more data sources. As examples, the input data may include product sales information, information regarding the communication frequency of a rep to an HCP, types of products promoted, or brand or marketing strategy rules. The market data may include sales numbers for products, company revenue numbers, sales statistics for particular HCPs, sales statistics aggregated from multiple HCPs, or pharmaceutical industry metrics. The strategies may include achieving quarterly sales targets, achieving quarterly revenue targets, or achieving quarterly profit targets. The analytics may include interaction data for specific pharma reps. The context may include contextual information that is specific to each channel, rep or HCP. The content may include customized content that is suggested to reps, and for targeting towards individual HCPs or segments. The campaigns may include advertising campaigns or product launch campaigns. The models may include sales models or forecasts or revenue models or forecasts.

The sources of the input data may include databases for customer relationship management (CRM), marketing automation systems, website target management systems, customer data platforms (CDP), market segmentation, sales, a pharmaceutical company's proprietary databases, and/or third party provided/generated data. In some instances, the input data may include field notes taken by reps in their interactions or visits with HCPs.

A plurality of temporal goals for attainment may be defined based at least in part on the input data. The plurality of temporal goals may be classified into at least two categories of different time scales. There may be at least 2, at least 3, at least 4, at least 5, at least 10, at least 15, at least 20, at least 30, at least 40, at least 50, or at least 100 temporal goals. There may be more than 2, more than 3, more than 4, more than 5, more than 10, or more than 20 time scales. The categories may include both long-term and short term. In some cases, the plurality of temporal goals comprise at least one long-term goal on a first time scale ranging from about six to twelve months, and a plurality of short-term goals on a second timescale that is daily, weekly, or monthly. The plurality of temporal goals may be expressed in a form of actions and/or responses to actions on the different timescales.

In some instances, at least one long-term goal may be provided on a first timescale. The first timescale may range from about six to twelve months. The first timescale may be at least 2 months, at least 3 months, at least 4 months, at least six months, or at least a year. A plurality of short-term goals may be provided on a second timescale. The second timescale may be daily, weekly, or monthly.

The CIE can be configured to select target, channel, content and timing to optimize the customer experience/journey (e.g. interactions/engagement between reps and HCPs). The CIE can be configured to process a plurality of OC action candidates including rules, campaigns and ML model outputs, which are subject to constraints, filtered and consolidated before being provided to an optimization model. The optimization model can be configured to take into account a number of factors (e.g. value, urgency, priority, cost, content affinity, and channel affinity) in selecting the appropriate target entity, channel, content and timing to optimize the customer experience/journey (e.g. interactions/engagement between reps and HCPs). The CIE is flexible and scalable, in that any number and type of channel, account and action candidates can be added or removed at any given time. The customer experience/journey is optimized for transparency, where the next best and alternative actions are presented to the actor types (e.g. reps), with explanations of the effects of those actions (having quantifiable component values), and aided by simulation and visual reporting to the actor types (e.g. reps and stakeholders).

The CIE may comprise the optimization model described above. The optimization model may comprise an objective function. A plurality of temporal goals and a set of constraints may be input to the optimization model. The channels can enable or facilitate communications between one or more actor types and the one or more target entities. The channels may include communicating with an HCP via email, short message system (SMS), telephone, web chat, in-person, or any other communication channel. There may be at least 1, 2, 3, 4, 5, 10, 15 or more types of communication channels.

In some embodiments, the one or more actor types may comprise one or more enumerated actors including sales representatives, district managers, or medical science liaisons that are associated with the promotion of the product to the one or more target entities. In other embodiments, the one or more actor types may comprise automated systems including marketing automation systems or web portal management systems.

The set of constraints may comprise action-type constraints, channel constraints, channel capacity constraints, pacing constraints, and/or channel fatigue constraints across a plurality of channels. An action-type constraint may include a restriction on an action or communication that can be performed by a rep when engaging with a HCP. A channel constraint may include a constraint on a particular communication method (e.g., email or text) between the rep and HCP. A channel capacity constraint may include a volume or frequency of communications that are routine/standard for each channel. A pacing constraint may include a timing or frequency by which the rep sends communications to a HCP. A channel fatigue constraint may include overuse of certain channels that may cause the rep and/or HCP to shun or shy away from those channels.

The optimization model can be used to generate a set of multi-dimensional actions with predicted action values for at least a subset of the plurality of channels. The predicted action values may collectively maximize an impact value of the objective function while balancing a feasibility of the plurality of temporal goals with respect to one another. The set of multi-dimensional actions with predicted action values may be generated based on one or more of the following: a value of the one or more target entities, a relative impact value of the actions, a probability of success of the actions, and a timing value of the actions. The predicted action values may include scores (e.g., of 1, 2, 3, 4, 5, 6, 7, 8, 9, or 10 out of 10), or probabilities, or combinations of scores and probabilities. The subset of channels may include at least 1, at least 2, at least 3, at least 4, or at least 5 of the channels. The balancing may be implemented based on the predicted action values.

The objective function may be configured to return a value function that is a product of a targeted entity value, action impact values, engagement probabilities, and success probabilities for achieving the plurality of goals, and time values of the actions.

The set of recommended user multi-dimensional actions may be designed to complement each other so as to enable cross-channel optimization across the subset of channels, based at least in part on an execution capacity of the enumerated actors, capacity limits set of the automated systems, utilization or consumption of content, or usefulness/relevance of the content.

The optimization model can be configured to iteratively search for actions to maximize expected action values by applying and varying the plurality of constraints and parameters to (1) influence the predicted action values and (2) monitor changes in the predicted action values. The optimization model can be configured to iteratively apply and vary a plurality of parameters to influence the predicted action values and monitor changes in the predicted action values. The optimization may apply at least 2, at least 3, at least 4, at least five, or at least 10 iterations. The plurality of parameters comprise one or more of the following: cost, impact, urgency, recency, channel propensity, physical proximity, representative (rep) engagement, time to engage, account priority, or factor priority.

The optimization model may be configured to search through a range of predicted action values for the plurality of channels when the plurality of parameters is being varied subject to the set of constraints. This may be performed to identify a subset of predicted action values that form a basis for the set of multi-dimensional actions.

The set of multi-dimensional actions with predicted action values for at least a subset of the plurality of channels can be provided to the CRM system to facilitate optimization of the customer journey/experience. Those actions with predicted action values can be used in field sales (e.g. remote calls by reps to HCPs, face-to-face meetings between reps and HCPs, and emails from reps to HCPs), by field medical personnel (e.g. MSLs and patient support), in marketing automation (e.g. HQ email, messaging to customers, mailers), or digital platforms (e.g. social media, web portals, or third party conferences/events).

FIG. 1B illustrates an example of the architecture in FIG. 1A. For example, the CIE can be configured to receive information/content from multiple channels, such as customer experiences, campaigns, and activity performance relating to rep-HCP interactions. A plurality of goals (e.g. long term and short term), targets, constraints and external influences may be provided to an objective function in the CIE. The CIE can be configured to generate campaign activations, experience activations and CRM suggestions to the reps for the multiple channels.

FIG. 2 illustrates examples of inputs that may be provided to the CIE for optimization. The inputs may be classified in a number of ways, for example by groups, influence labels, and dimensions. Examples of types of groups may include account, use case (i.e., factor), channel, date/time, and end user.

The inputs in each group can have difference influence labels and dimensions. For example, the influence labels for the inputs in the Account group may include account priority (long term), account interaction urgency (short term), and segment priority. Each input may be associated with one or more dimensions. For example, in the Account group, the dimension for account priority (long term) may be account, and the dimension for segment priority may be segment.

Referring to the Channel group as another example, the inputs may have influence labels including action impact score, channel propensity, interaction recency (time lag), and interaction frequency (rolling sum). Each of those inputs can have multiple dimensions. For example, the dimensions for action impact score may be account/channel, and the dimensions for interaction frequency (rolling sum) may be account/channel/date.

For use case (i.e., factor) group, the influence label may comprise use case base action value, which corresponds to action candidate dimension; and use case criticality, which corresponds to action candidate dimension. For the date/time group, the influence label may comprise: account availability, which corresponds to account/date dimension; time-dependent HCP engagement propensity, which corresponds to account/date/channel dimension; and date influence, which corresponds to account/date/action candidate dimension. For the end user group, the influence label may comprise: rep engagement propensity, which corresponds to account/rep/channel/source dimension; and location proximity, which corresponds to account/rep/date dimension.

The sources of the inputs may include any of the data sources described elsewhere herein. For example, the sources may include marketing automation triggers and external data feeds. The sources may also include a module that monitors/tracks the pacing or frequency of interactions between reps and HCPs. In some instances, the sources may include a user interface comprising a customer configuration panel that allows actor types to build/customize engagement plans with different HCPs or segments.

It should be noted that not all of the inputs shown in the table of FIG. 2 have to be provided to the CIE at the same. Different inputs (or combinations of inputs) can be selectively provided to the CIE. For example, a subset of inputs may be utilized at time period A, and a different subset of inputs may be utilized at time period B. There may be overlaps between different subsets of inputs.

The inputs may be scored by a ML module, simple scoring rubrics, may include customer-provided scores, or may be disabled. These inputs may have different tiers: for example, an influence label of account priority may indicate this is a drive targeting.

An example of a simple optimization is as follows. For example, there may be certain rules, such as an action impact score may indicate that visits are worth four-times (4×) of emails. An algorithm may be used to determine account availability, for a particular account at a particular date/time. Account priority (long term) may be considered when determining which tiers/deciles to drive targeting efforts.

A more complex optimization example may involve multiple inputs and use of one or more algorithms. For example, a customer marketing mix analysis may consider account priority (long term), segment priority, and action impact score when performing the analysis. A customer brand strategy process may consider account priority (long term), segment priority, and action impact score when performing strategizing actions. A ML algorithm may consider channel propensity, account availability, date influence, rep engagement propensity, and location proximity when making recommended action sets. Alternatively or additionally, another algorithm may consider channel propensity, account availability, date influence, rep engagement propensity, and location proximity when making recommended action sets. A marketing automation influence module may consider channel propensity, account availability, date influence, rep engagement propensity, and location proximity when calculate marketing automation influence.

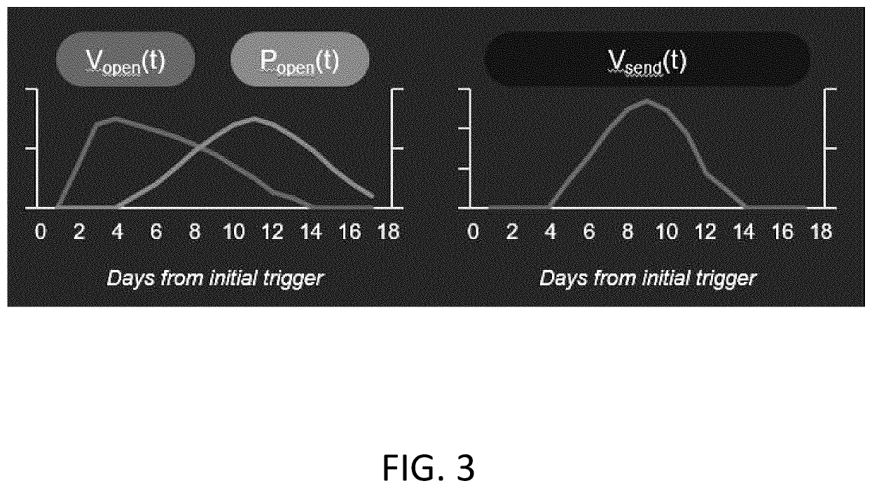

FIG. 3 illustrates an example of email timing optimization according to some embodiments. It shows a simplified version schematic of the full value function. It should be noted that the optimization is highly configurable, depending on the type of data and amount of data. The underlying context may be obtained from a custom model which indicates that a HCP should receive message ‘X’ in the coming week. The CIE can be configured to calculate the probability of an email (containing the message ‘X’) being opened by the HCP. The probability can be calculated for each day that the email can potentially be sent.

The CIE can be configured to perform an optimization of the email timing. For example, the objective function in the CIE may include a value function. The value function may be defined by the product of a probability value with an expected value:

V_{send}(t)=Pₒₚₑₙ(t)*Vₒₚₑₙ(t)

where V_{send} (t) is the expected value of an email sent by a rep to a HCP (at time t). Pₒₚₑₙ (t) is the probability of the email being opened by the HCP (at time t), Vₒₚₑₙ (t) is the expected value of the email being opened (at time t), and the time t is a variable.

As shown on the left of FIG. 3, the plots for Vₒₚₑₙ (t) and Pₒₚₑₙ (t) are shown as two separate profiles on the chart, as a function of number of days from initial trigger (for example, an event, a visit, etc.) with the HCP. The right of FIG. 3 shows the profile for V_{send} (t), which is a product (combined plot) of Vₒₚₑₙ (t) and Pₒₚₑₙ (t). As shown on the right of FIG. 3, the optimal timing for the rep to send the email to the HCP is about 8-10 days from the initial trigger. The optimal timing is calculated to ensure the best chances of successful interaction between the rep and the HCP. The optimal email timing can be suggested to the rep, for example through a user workflow interface.

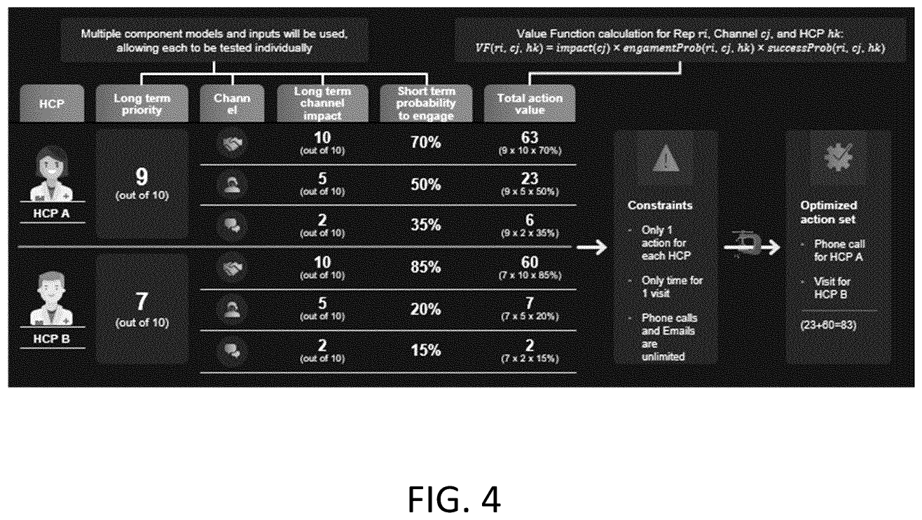

FIG. 4 illustrates an example of the optimization of rep interactions with different HCPs (e.g. HCP A and HCP B). The CIE can be configured to combine or average various machine-learned metrics to produce an optimized set of actions for a pharma rep to engage with a HCP. The objective function may be a type of weighting or averaging method used by the CIE, such as a weighted sum, product, or average.

Multiple component models and inputs can be used to calculate a value function. For example, factors such as long term priority, long term channel impact, and short term probability to engage may be considered for each of a plurality of different channels.

Each HCP may be assigned a long term priority score. For example, HCP A may have a long term priority score of 9 (out of 10), and HCP B may have a long term priority score of 7 (out of 10). In other words, HCP A has a higher longer term priority value than HCP B. The channels for reps to communicate with the HCPs may include in-person meetings, telephone calls, and/or email or text messaging. The long term channel impact is quantified as a score for each channel. For example, in-person meetings have a long term channel impact score of 10 (out of 10), whereas emails/text message may only have a long term channel impact score of 2 (out of 10). The short term probability to engage may be quantified as a percentage or probability value. For example, the short term probability to engage for HCP A over in-person meetings is 70%, whereas the short term probability to engage for HCP B over in-person meetings is 85%.

For each channel, a total action value may be calculated. A value function calculated for a particular Rep ri. Channel cj, and HCP hk may be as follows:

VF(ri,cj,hk)=impact(cj)×engamentProb(ri,cj,hk)×successProb(ri,cj,hk)

The total action value is a product of the long term priority score, the long term channel impact score, and the short term probability to engage. As shown in FIG. 4, the total action value for HCP A over in-person meetings is 63, while the total action value for HCP A over emails/text messaging is only 6. The total action value for HCP B over in-person meetings is 60, while the total action value for HCP B over emails/text messaging is only 2.

The short-term propensity to engage may be correlated with the long-term channel impact, where higher propensities to engage may be associated with higher long-term channel impacts. After the total action values have been calculated, the CIE is configured to apply a set of constraints to produce an optimized action set for the rep. The set of constraints may include, for example, limiting to only action for each HCP, limiting the time for only one in-person visit, while the number of phone calls and emails are unlimited. Based on the constraints and the total action values, the optimized action set for a rep may comprise making one phone call for HCP A (which has a total action value of 23), and one visit for HCP B (which has a total action value of 60). Accordingly, the total value score of the optimized action set for both HCP A and HCP B is 83 (60+23).

The value function may be a function that produces an output based on quantitative values of the predictions. The output may be a weighted sum, an average, or a product of the quantitative values. For example, scores indicating to what extent a particular HCP is a valued long-term customer, a long-term impact of repeatedly performing a particular type of communication with the HCP rep, and a short-term probability of the HCP to engage with a pharma rep for a particular communication, may be multiplied together to determine a value for that action. For example, the value function may determine a value of emailing a high-priority HCP who is not likely to respond to email to be low. Value functions may be calculated for many different types of actions and compared to one another to determine optimal actions.

To each value function for each particular action, the CIE may apply constraints. Constraints may include restricting the number of actions able to be taken per HCP, the number of time available for a rep to contact an HCP, or the frequency of communications that may be provide to an HCP. After these constraints are applied, the CIE may determine a set of optimal actions. The optimal actions may be provided to a customer relationship management (CRM) platform.

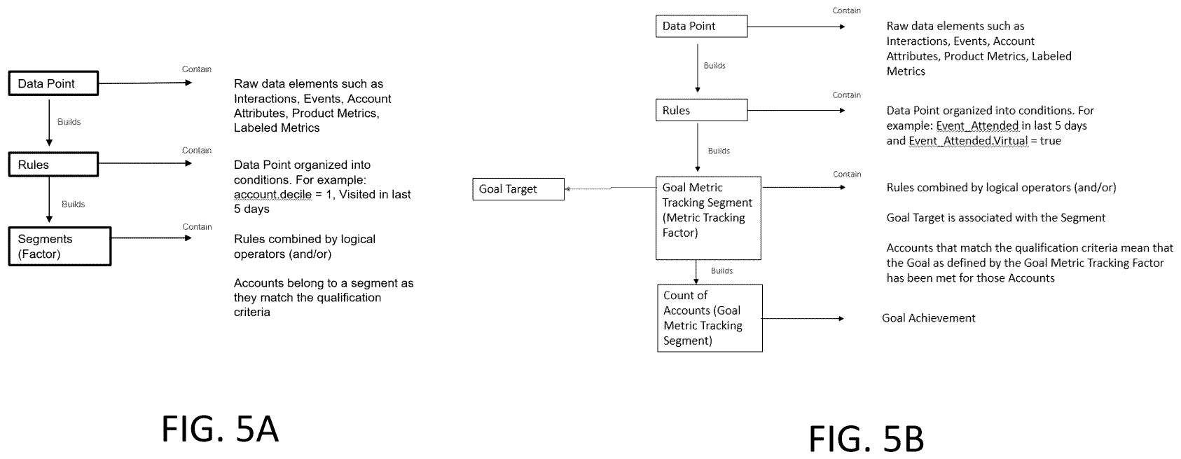

FIG. 5A is a flowchart showing the manner in which data points, rules and segments are built upon based on different campaign segments/goals. Referring to FIG. 5A, a plurality of data points may be received and pre-processed. The plurality of data points may comprise raw data elements including interactions, events, account attributes, product metrics, and/or labeled metrics. Next, the data points may be used to generate a set of rules. The set of rules may comprise an organization of the plurality of data points into conditions. For example, accounts may be split by deciles having a numerical value (e.g. 1) and previous action (e.g. visited in last 5 days). Next, the set of rules may be processed to generate a plurality of segments. For example, different rules can be combined in a variety of configurations using logical operators. Next, an account is determined as to which of the plurality of segments the account belongs to, based on whether the account matches a qualification criteria for each of the plurality of segments. The accounts may be categorized into different segments. For example, a group of accounts may belong to a same segment if they match a qualification criteria for the segment.

FIG. 5B shows a goal metric tracking segment model, which is similar to FIG. 5A. In FIG. 5B, different rules can be combined in a variety of configurations using logical operators for a goal metric that tracks by segment (i.e, a goal target is associated with a particular segment). Accounts that match the qualification criteria may indicate that the goal (as defined by the goal metric tracking factor) has been met for those accounts. A count of the number of accounts matching the qualification criteria provides a quantification/substantiation of the goal achievement.

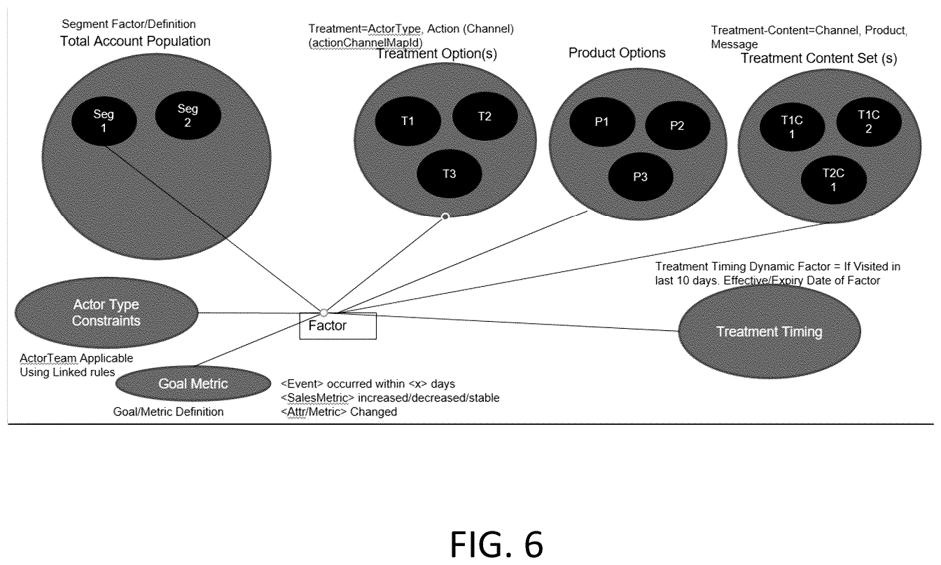

FIG. 6 is a schematic showing the types of inputs that are provided to the CIE for optimization. It shows a general framework for ensuring that rules are incorporated as constraints to the CIE. The inputs may include total account population, treatment option(s), product options, treatment content set(s), and treatment timing. Goal metrics may include any of the long term and short term goals as described herein. Actor type constraints may include the constraints on rep availability/resources as described herein. The CIE can be configured to identify the optimal treatment options, timing and content sets for each segment (HCP), across one or more product options, so as to achieve the goal metrics subject to actor-type constraints. Examples of constraints may include resource capacity, account assignment constraints, product authorization constraints, periods in which a rep may be unavailable, work week schedule for a rep, and whether an actor type is a person or an automated system. The suggested optimal actions may be delivered to the rep through a CRM system. MAS or a custom platform. The actions may span multiple channels (e.g. email, letter, survey, face-to-face meetings, virtual meetings, phone calls, chat/text messaging, meeting at a healthcare facility, or meeting at an external event such as a conference). The actions may also span different geographical areas (e.g. regional or national). Each channel may have a min/max capacity per segment (e.g. minimum and maximum number of communications that can be sent over a time period, for each channel to each segment). Channels may have constraints. For example channel-type constraints may include time constraints, whether the messaging/updates can be asynchronized, as well as time/distance constraints if the channels involve physical traveling between locations. In some instances, the actions may include a primary action, a sub action, a follow up action, or a sequence of actions. The actions may have different values (e.g. expected values as described herein) and vary in duration.

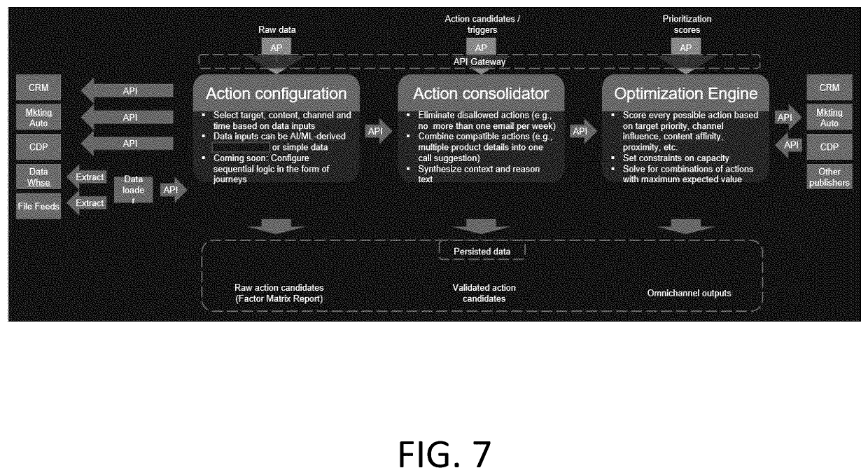

FIG. 7 illustrates a system that is configured to interface with external models and analytic access points. The system may comprise an action configuration module, an action consolidator, and an optimization engine. The optimization engine may be the same or similar to the CIE described elsewhere herein. The action configuration module, action consolidator, and optimization engine can be configured to interface with external models and analytic access points through a plurality of using an application programming interfaces (APIs). The external models and analytic access points may include sources of data (e.g. CRM systems, marketing automation systems. CDPs, data warehouses, and file feeds). A data loader can be used to extract data from the data warehouse and file feeds. The action configuration module can be configured to receive and process raw data. The raw data inputs can be simple data or AI/ML-derived (for example, using natural language processing-NLP). The action configuration module is configured to select a target (e.g. HCP target), content, channel and time based on the raw data inputs, to generate a plurality of raw action candidates. In some embodiments, the raw action candidates may be provided as a factor matrix report. The action consolidator is configured to receive and process the raw action candidates. For example, the action consolidator can eliminate disallowed actions (e.g. no more than one email per week to be sent to a HCP), combine compatible actions (e.g. multiple product details can be consolidated into a single suggested call to a HCP), and synthesize context and reason text to generate validated action candidates. The optimization engine can be configured to receive the validated action candidates, and generate scores for the list of actions based on target priority, channel influence, content affinity, proximity, etc. Constraints are then applied, and the optimization engine is configured to solve for combinations of actions with maximum expected value over multiple channels (omnichannel outputs). The omnichannel outputs of the optimization engine can be provided to the actor types via CRM, marketing automation, CDP, or any other types of publishers or publishing services.

The CIE platform can take into consideration costs to enable value management, which is the principle and practice of managing every decision (both in planning contexts and in daily decision-making) as aided by the CIE platform so as to maximize economic value. The CIE platform can use or apply any cost number or cost function for any channel within multiple channels (OC).

In some embodiments, the cost may be set to be greater than zero. The CIE platform can calculate and report the return on investment (ROI). A ROI threshold can be used. The CIE platform can also calculate the expected net benefit and apply a threshold to it.

In other embodiments, the cost may be set to zero for certain channels. In such embodiments, the CIE platform does not report on ROI for those channels and ROI thresholds are not used. Nonetheless, the CIE platform can still calculate the expected benefit.

A channel as described herein is a mode of contact involving reps. The cost can vary significantly by channel, for example a rep is making an in-person visit, a phone call, or sending an email to a HCP. The CIE platform can assist with sales operations, by performing action optimization based on expected net benefit (the cost will be greater than zero since it involves field operations). Separately, the CIE platform can also assign reps based partly on rep capacity, where the costs for various modes of contact (or channels) are measured as a function of the amount of time to be spent. The separation/decoupling of the action optimization from the rep assignment is a key design feature of the CIE platform, which enables efficiency and modularity of the platform.

For digital channels that do not require in-person or field operations, the channel contact cost can be set to zero. Either simple or complex cost models can be used, using a hybrid of fixed and/or variable costs. The cost of excess contacts can be modeled and used, which can facilitate recognition of customer impact costs, balance AI-driven actions with rule and binary constraints to control excess contacts, and transition to an AI-optimization driven decision making process.

The following is an example of how the CIE platform can incorporate cost into the value framework. The CIE platform can calculate a net expected value by subtracting expected cost from expected value. The expected cost may be calculated based on contact cost and engagement cost. The contact cost can be multiplied by the probability of a rep attempting contact with a HCP. The engagement cost is based on the cost to the rep to act on a suggestion. The contact cost and engagement cost can be provided to the CIE platform as either discrete numbers or formulas (cost models) that may depend on one or more additional factors. For example, if a rep attempts to contact a HCP an excessive number of times within a short time period via a channel, there may be additional costs in the form of customer/HCP annoyance (or perturbation) cost, which may further reduce the net expected value.

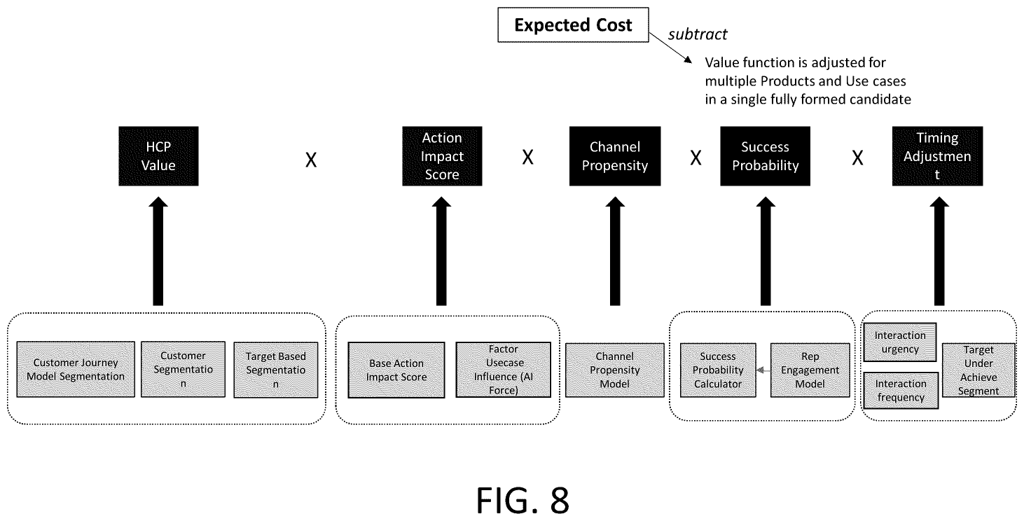

FIG. 8 shows the calculation for the expected action value for each fully formed suggestion candidate in an example embodiment. The action value can be calculated using the value function described herein. Referring to FIG. 8, the expected action value is given by the product of HCP value, action impact score, channel propensity, success probability, and timing adjustment. The value function can be adjusted for multiple different products and use cases in a single fully formed suggestion candidate.

The HCP value may be calculated based on segmentation, for example customer segmentation, target based segmentation, etc. Segment types may be assigned weights, and valuations applied to segments. For example, a value may be applied for each segment and weights for each segment type to derive an overall HCP value. The expected cost can be factored in by subtracting the expected cost from the overall value.

As described previously with reference to FIG. 5B, factors can be associated with a goal (goal metric tracking factor). The success probability can be calculated per goal and action, using the time lag between an action triggering event and a goal metric measurement. The success probability can be measured periodically with a time lag over a time period. For example, a success probability achieved at time (t) can be fed back into the optimizer (e.g. CIE) by applying suggestion candidates associated with the goal, as to which actions have better success probability at time (t) for a particular campaign. The feedback can be used adjust the actions for a population targeted, to compensate for the difference between targeted versus achieved goal success probability at time (t).

The CIE described herein (containing AI-based models, machine learning models, etc.) can be combined with rules, for example business-type rules or campaign rules. The combination/infusion of business-type rules or campaign rules to AI-based/machine learning models is a unique modification, in that conventional models typically do not consider those type of rules when analyzing data and/or making predictions. The above provides a practical aspect and improves the utility/accuracy of those models.

The combination of rules with AI-based models is illustrated in at least two of the following aspects which are related. The first aspect is the value function described herein. The value function may comprise components can be used to make adjustments to the value estimate (or action value). The adjustments can be based on various conditions. For example, if a suggestion to a rep (that is under evaluated) is based a rule, the suggestion would then receive an upward adjustment in its action value to reflect the suggestion as a top priority. The CIE is configured to make those adjustments within the value function.

The adjustments may be made across a scale, and a specific adjustment may be a scaled number. The scale may range across any numerical range (e.g. between one and five), or qualitative (e.g. low, medium, high). In some instances, the scale may include absolutes. For example, if a suggestion is based on a rule and the rule is marked as absolute, the suggestion (which may contain a suggested action) has to be executed by a rep regardless. On the other hand, if a suggestion is not based on an absolute rule, the suggestion may be subject to upward/downward adjustments in the value function, such that the suggestion has a higher/lower likelihood to be selected, but is not guaranteed to be always selected (or selected by default). The above is an example of how rules can be integrated into the CIE (AI-based/machine learning models).

The second aspect of the combination of rules with AI-based models can be illustrated on the frontend. The degree of control above (scaled adjustments, or absolute/low/medium/high), and priority given to each rule, can be implemented via a user interface (UI). The UI can be configured to enable/allow users control of the assignment of the rules, and integration of those rules into the AI-based/machine learning models. The rules may be defined for example by one or more users (e.g. marketing manager, sales managers, etc.) or members of their team. In some embodiments, the UI can include buttons or sliding bars that allow the users to easily control the scale, either quantitatively (e.g. between one to five) or qualitatively (e.g. absolute/low/medium/high).

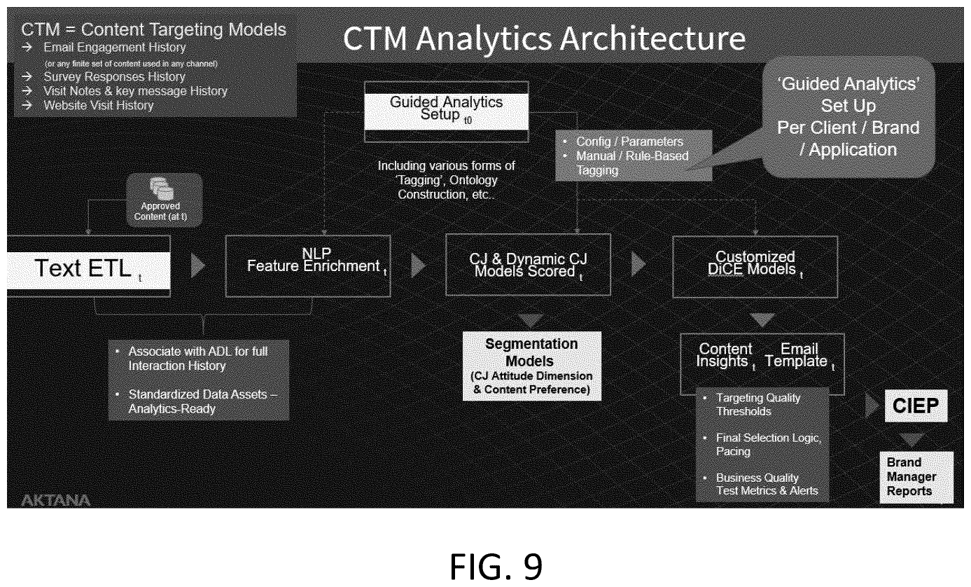

FIG. 9 illustrates a content targeting models (CTM) analytics architecture according to some embodiments. This architecture is configured to facilitate content management in content targeting using AI. For example, suggesting to reps what are the appropriate or impactful content, in different contexts, to target towards different HCPs.

One example of a use case includes which email template to assign to an email suggestion. For example, there may be multiple options about which email content to use, and/or which templates should be assigned to each email with subject line to use. Other use cases and examples of content may include product brochures, flyers, informational posters. CDs, DVDs, samples, etc. relating to the products or certain health conditions. Those content may be produced by the marketing department of a pharma company. Content can also include links to relevant websites containing text, images and/or video about the product, health conditions. Content can include any information or topics that are of potential interest/value/benefits to the HCPs, or an invitation to discuss those topics or go over certain information for additional insights. Accordingly, a rep may have multiple options on which content to share with or relay to HCPs, and how to provide content in a targeted manner that is useful and valuable to the HCPs to consume.

The above content contains a variety of different data, and depending on the data type, there may be variations in the use cases (e.g. how the content is being relayed or communicated by the reps to the HCPs). Data can contain content, as well as history of prior interactions with content (by both HCPs and reps). For example, a website visit history is an interaction with content, for example which webpages a HCP visited, length of time the HCP spent on the page/website, forwarding/sharing of a link to the website by the HCP to others, etc.

Pharma companies often generate surveys for both reps and HCPs. The surveys can include questions that the reps are answering about certain HCPs. In some instances, the surveys are directed to the HCPs, to solicit the opinions and attitudes of HCPs towards certain products or actions. The responses to surveys are akin to interactions with content (the surveys being the content itself). When reps visit HCPs, they often take notes before and after the meetings. The notes can contain useful information about the interactions and general attitudes/responses of the HCPs towards the topics discussed or content presented. One use case for surveys is suggesting to the reps a series of questions, and which questions to ask next to the HCPs. The surveys can be dynamic or at a predefined interval, for example when the reps meet the HCPs or after they meet with the HCPs, the reps may answer questions about the HCPs, which questions were asked to the HCPs, the HCP responses to those questions, which questions were answers, which questions were not answered, etc. The general objective is for the reps to ask questions that maximize the value of information that can be extracted from responses to those questions, and to identify those questions for the reps (which is a capability of the CIE described herein).

As described herein, data can include content, responses to content, interactions with content, etc. The data from various databases often comes in complex structures. To facilitate processing, the data can be reduced to a data structure—ADL (analytic data logs)—which can then be used to develop models that are later deployed. As described herein, ADL is a family of data assets including at least three distinct table complexes: (1) ADLx which are tables of event/interaction level history, (2) ADLHs which are tables of HCP-level features, and (3) ADLs which are actor and HCP state tables with details and summaries of recent events for reps and HCPs. Data fidelity is ensured in that the data used for model development is the same data that is used in production to execute the models later. This can help to reduce quality control issues relating to implementation of earlier models.

Referring to FIG. 9, a database may comprise a history of approved content items that have been curated/approved. Next, an extract, transform, load (ETL) process is used to extract the underlying text from the approved content. Next, the extracted text is processed using natural language processing (NLP). The NLP may comprise a set of NLP algorithms or augmentations that have feature enrichment capability. The NLP can be configured to perform sentiment analysis on the extracted text, extracted topics, etc. The NLP may include an ontology containing a list of relevant words, and associating those words in the text that have semantic similarity to the words in the list.

The extracted raw text and the NLP-processed text can be associated with analytic detection logs (ADLs) to obtain a full history of content interactions, and further standardized and organized into a structure/format that is suitable for subsequent analytics in the CTM analytics architecture. Accordingly, for each HCP, there is enriched data containing the history of the HCP's interactions with content, and other information about the content from semantic analysis.

Next, once the standardized data assets are ready for analytics, they are fed into machine learning models, which include for example customer journey (CJ) and dynamic CJ models. The models can be configured to calculate one or more probabilities. For example, a probability of a HCP responding to an email template based on the subject line, a probability that based on the data received, the HCP is likely to be comfortable prescribing a drug or starting a patient on a course of therapy, and others. The models can be customized and used to determine content targeting.

In some instances, there may be a guided analytics setup, which allows the content to be tagged. Tagging may include tagging emails from the reps, tagging emails from the HCPs, tagging visit notes in which a rep indicated that the HCP is likely to be comfortable prescribing a drug, or tagging a dataset with a high level description of its content. A tag may indicate that an email relates to setting up a meeting with an expert. Another tag may indicate than an email is about patient compliance material. Another tag may indicate that an email is about dosing. The content items can be tagged in ways that are useful for business analytics. The content can be tagged by relevant personnel, for example a scientist or business analyst at the pharma company. In some instances, the tagging of the content may be partially or fully automated. The tagging may be subsequently reviewed and curated if necessary, by subject matter experts and/or customers, to meet a company's business needs and also for regulatory compliance.

A dynamic CJ model can be configured to detect changes in attitudes of HCPs over time, hence the dynamic qualifier. The changes are based on the HCPs interaction history. For example, a HCP may be previously hesitant in prescribing a drug to a patient, but changed to being comfortable prescribing the drug. This data and interaction history can be used to predict which HCPs (and other HCPs in the same or similar segment) are likely to change their attitude about a drug, for example from being less comfortable to more comfortable prescribing a drug, or initiating new patients on treatment course, etc.

Next, the results (e.g. scores or probabilities) from the CJ models and dynamic CJ models are fed into Diverse Counterfactual Explanations (DiCE) models. The DiCE models can be used to predict the probability of a HCP being comfortable in prescribing a drug had their communication history been different (for example, if the HCP had responded differently to a survey question in the past). The counterfactual indicates the probability of the outcome, the outcome being whether the HCP is likely to prescribe the product. The objective of calculating the counterfactuals is to determine which actions or responses led to different outcomes in the past, such that the probability of a future desired outcome is higher (e.g. a HCP being more likely to prescribe a drug). Once the counterfactuals have been determined, the information can be converted to a topic recommendation for the rep to communicate to the HCP. In other words, determining which are these counterfactuals, and inferring from them what content/topics a rep should be discussing with the HCP. The counterfactuals can also be used to infer which email templates should be assigned to which emails based on their subject line and/or content.

Next, the output of the DiCE models (e.g. content insights, email template) is managed, for example by applying a final selection logic that controls the pacing and frequency at which reps should communicate the topics and content to HCPs.

As described elsewhere herein, the data sources can vary, and there are variations in use cases. Taking as an example where the data sources include one or more of the following: (i) survey questions and answers from reps and/or HCPs. (ii) emails (body text. URLs embedded therein, subject lines, etc.) and history of HCP responses to emails from the reps. (iii) key messages associated with rep visits to HCPs, and (iv) visit notes taken by reps before and after visits to HCPs. Additional data sources may include HCP website visit histories (including the content associated with pages visited), and additional content associated with key messages, event invitations and rep visits to HCPs (such as posters, DVDs and CDs, brochures, etc. that the reps provide to the HCPs). Each of the above data sources, alone and in combination, can provide the basis for a multitude of AI-supported use cases using the CTM analytics architecture shown in FIG. 9.

For example, a first use case may include one or more of the following: (i) selecting optimal email content (template, subject line) for a rep to send to a HCP, (ii) recommended topics for discussion between the rep and a HCP, (iii) segmentation of HCPs based on content affinity, and (iv) augmenting of CJ analytics with information relevant to the attitudes of the HCPs. The first use case may be based, for example on data sources comprising of emails.

A second use case may include one or more of the following: (i) recommended topics for discussion between the rep and a HCP, (ii) segmentation of HCPs based on content affinity, and (iii) augmenting of CJ analytics with information relevant to the attitudes of the HCPs. The second use case may be based, for example on data sources comprising of rep visit notes and/or key messages (content associated with key messages).

A third use case may include one or more of the following: (i) recommended topics for discussion between the rep and a HCP, (ii) segmentation of HCPs based on content affinity, (iii) augmenting of CJ analytics with information relevant to the attitudes of the HCPs, and (iv) recommended questions to ask reps and/or HCPs in an on-going survey, or ways to reduce a large survey to a smaller and more compact/effective survey. The third use case may be based, for example on data sources comprising of survey questions and answers from reps and/or HCPs.

A fourth use case may include recommending one or more items for use via one or more channels, the items being selected from a list of available content items. The fourth use case may be based, for example on data sources comprising of available content relevant to any given channel. Examples may include (1) webinar invitations when there are multiple webinars planned. (2) content packages that are made available to reps to bring to visits with HCPs, such as samples, brochures, posters, DVD/CD, etc.

A fifth use case may include one or more of the following: (i) recommended topics for discussion between the rep and a HCP, (ii) segmentation of HCPs based on content affinity, (iii) augmenting of CJ analytics with information relevant to the attitudes of the HCPs, and (iv) recommended content for targeting on a pharma company's website. The fifth use case may be based, for example on data sources comprising of the HCPs' website visit histories.

FIGS. 10-1 and 10-2 illustrate a behavior simulator in accordance with some embodiments. The behavior simulator can be used as a testbed to simulate the behavior of the CTM analytics architecture (and also any of the systems described elsewhere herein), before the systems are publicly launched to customers. The simulations of the behavior over a time period (e.g. 30 days) can help to ensure that the systems perform as expected/desired, prior to going live.

The behavior simulator comprises a plurality of control parameters that can be used as levers/dials to make granular adjustments to the behavior of the system. Examples of those parameters may include return on investment (ROI), certain thresholds, matrix parameters, constraints at different levels, etc. The control parameters can be adjusted to determine the behavior of the system, along with actual empirical data about HCPs and machine learning models.

The behavior simulator enables the system to be run end-to-end with different settings/parameters to observe the results/behavior of the system over a period of time (e.g. on a first day, second day, third day and so forth), whereby the behavior of the system on a next day is dependent on the system's behavior on the previous day. The dependency is based on various interactions occurring (or not occurring) on the previous day. The reps and HCPs' interactions with the system on a particular day often affect the outcome on the next day or following days. For example, a HCP may or may not respond to a rep's suggestion or outreach. For instance, a HCP may respond to a rep's email, another HCP may open the email but may not respond, another HCP may open the email but may not click on a link in the email, another HCP may ignore the email, and so forth. Multiple scenarios of the types of interactions may be possible. The behavior simulator is configured to simulate the behavior of reps and HCPs virtually in an iterative feedback loop over N number of days, where N may be any integer greater than one that is defined by a user of the behavior simulator.

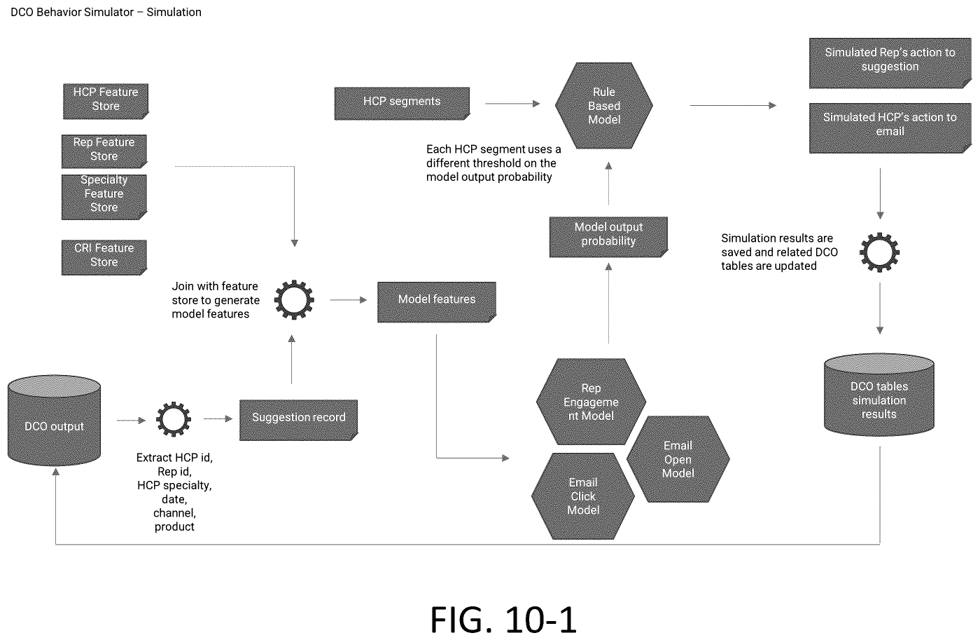

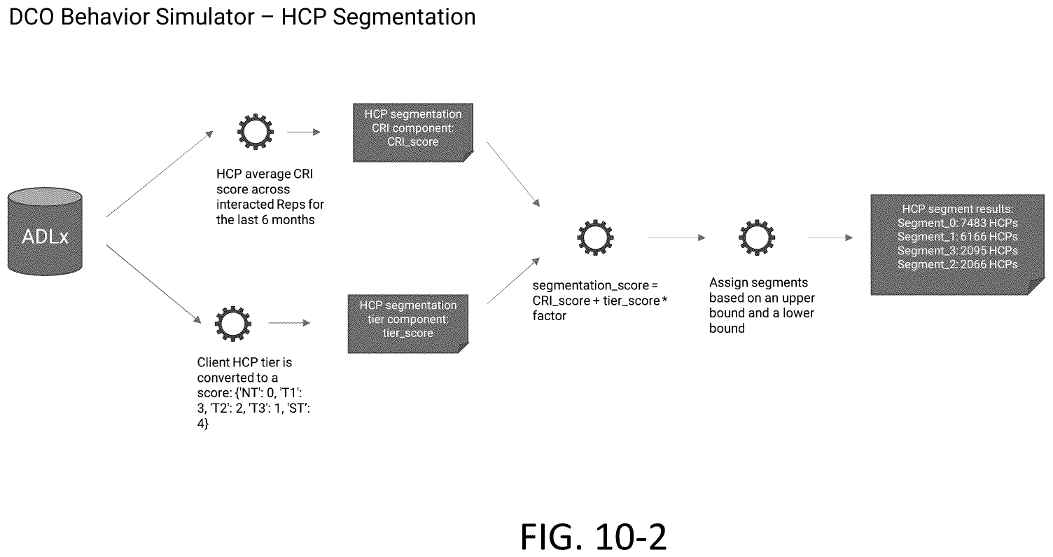

Referring to FIG. 10-1, the data sources for the behavior simulator may include a HCP feature store, rep feature store, specialty feature store, and customer relationship index (CRI) store. The HCP feature store comprises datasets about various HCP entities. For example, the datasets can comprise monthly updated HCP email/visit counts, rates, trends, etc. The rep feature store comprises datasets about various sales reps. For example, the datasets can comprise monthly updated rep email/visit counts, rates, trend, etc. The specialty feature store comprises data sets about the healthcare specialty areas of the HCPs and/or the specialty skillsets/knowledge of the reps. For example, the datasets can comprise monthly updated specialty email/visit counts, rates, trends, etc. The CRI store comprises CRI scores. For example, the datasets can comprise monthly updated CRI scores. The CRI is described in PCT/US2021/019469 (titled “Systems and Methods for Enhancing Suggestions with an Effective Index”), which is incorporated herein by reference in its entirety. The datasets in each of the above stores can be structured in a manner that is suitable for analysis and automated simulation by the behavior simulator.

The DCO (dynamic constrained optimization) output is the output from a first day of simulation. From the DCO output, information such as HCP identity, rep identity. HCP specialty, date, channel, product, etc. is then extracted into a suggestion record comprising of suggestions generated by the behavior simulator.

Next, the datasets from the data sources are joined with the suggestion record to generate a set of model features, which is a list of features used in the models (generated in model training). Next, the model features are fed into a plurality of models in the simulator. The models can include a rep engagement model, email click model, email open model, and various other models. Each model is indicative of the type of interaction behavior. For example, the rep engagement model can be used to predict a rep's responses to suggestions made by the system. The email click model can be used to predict the HCPs clicking on links within emails, and the email open model can be used to predict the HCPs opening and reading emails. The models are configured to generate probabilities of whether the above corresponding events are likely to occur. It should be noted that emails are used as an example of the types of models, and that the models can include responses to various other communications (and need not be limited to only emails). As examples, another model may relate to a rep's invitation to a HCP to connect virtually or through a phone: another model may relate to a rep's invitation to a HCP to attend a conference or speak at the conference. Accordingly, the models can be used to model any behavior of the HCPs in response to different interactions with the reps.

The probabilities from the model (model output probability) is then fed to a rule-based model. The probability may range anywhere from zero to 100%. The rule-based model may comprise any of the rules as described elsewhere herein. The rule-based model is also configured to receive HCP segmentation information. Each HCP segment may be associated with a different threshold based on the model output probability. The rule-based model is then configured to convert the model output probability with the HCP segmentation information into behavioral indicators. The model output probability (from a range of zero to 100%) are converted into the behavioral indicators which are in a binary format (1 or 0; yes or no). The behavioral indicators may include a simulated rep's action to a suggestion provided by the model, and a simulated HCP's action to a communication from the rep. As an example, a behavioral indicator may indicate whether a HCP has accepted (or not accepted) a suggestion given by a rep. Another behavioral indicator may indicate whether a rep has accepted (or dismissed) a suggestion that was recommended to the rep by the system. Accordingly, the behavioral indicators can be used to quantify whether an action has (or has not) occurred, by converting probabilities into a binary output (yes or no; 1 or 0)) that is indicative or reflective of the decisions/actions of both reps and HCPs.

Next, the behavioral indicators are saved and updated in the DCO database as tables of simulation results. Those results for a first day are then provided to the DCO on the left of FIG. 9 as data for a second day of simulation. The simulation can continue and iterate over multiple days (e.g. over 30 days), with each day's simulation iteratively building upon the previous day(s)'s simulation results.

Referring to FIG. 10-2, additional details about the HCP segmentation in the behavior simulator are next described. ADLX corresponds to an analytic detection and transaction logs, and serves as a data source for machine learning models. The HCP segmentation can be based on at least on a CRI component and a tier component. The CRI is the customer relationship index as described elsewhere herein, and comprises CRI scores. The tier component divides targets (e.g. HCPs or HCOs) by tiers, with each tier having a score. It should be appreciated that the HCP segmentation can be based on any scheme that is defined by or is suitable for a customer (e.g. a pharma company).

The CRI is a measure of a degree or strength of the relationship between a rep and a HCP, between a HCP and a company's brand, and relationships between relevant actor types and entities.

Each segment may be assigned a score. Next, the segments may be assigned based on an upper bound threshold and a lower bound threshold. The results of the HCP segmentation may include the number of HCPs in each segment.

Referring back to FIG. 10-1, once the HCP segmentation is completed, each segment for each behavior is associated with thresholds. The thresholds are used in converting probability values to binary values (0 or 1). If a probability of an event occurring (e.g. a HCP responding to an email) is above a threshold, the behavior simulator will convert the probability to a behavioral indicator corresponding to a yes or a 1 (positive occurrence, binary). Conversely, if a probability of an event occurring (e.g. a HCP responding to an email) is below the threshold, the behavior simulator will convert the probability to a behavioral indicator corresponding to a no or 0 (negative occurrence). The thresholds can be adjusted and set by a user. For example, a user can adjust the thresholds to simulate an environment in which the reps have high engagement with the HCPs, or to simulate another different environment in which the reps have low engagement with the HCPs, or to simulate another different environment in which the reps have medium engagement with the HCPs. The thresholds can be numerical values, or based on a sliding scale (low/medium/high). The machine learning models (e.g. rep engagement model, email click model, etc.) are trained with each day's simulation output results, and over time will more closely simulate the behavior of the HCPs and reps.

Snooze suppression is a feature that can be implemented in any of the systems herein, for example in the CIE or the behavior simulator. Snooze suppression provides an intelligent and productive way to suppress/avoid sending the same repeated suggestions to reps. Oftentimes, a rep may ignore or dismiss many of those suggestions, perhaps due to low relevance or impracticality given the existing circumstances. For example, a rep may be physically located far away from a HCP, and a suggestion for an in-person meeting may not be practical. A snooze suppression model is a machine learning model that can be configured to detect and monitor which suggestions were declined, ignored or dismissed by the reps, and the number of times those were declined/ignored/dismissed. The snooze suppression model can also predict which suggestions are likely to be suppressed the next day, following day, etc. Accordingly, there is a list of suppressed suggestions for each day, that is filtered out and not considered in the optimization process. This can help to improve and make the suggestion optimization process more efficient. Accordingly, the snooze suppression model can be used to determine, for each day, which suggestions should be suppressed based on past recent suggestions that are ignored or dismissed. The suppression list can contain different lists of suppressed suggestions that are specific to different reps, HCPs and segments.

### Rep Assignment Model

Next, a rep assignment model for the CIE is described. The rep assignment model can be used to optimally assign or match the appropriate reps to different HCPs, before suggestions are provided to the reps for performing certain actions over different channels (e.g. email or in-person visits to HCPs). Part of the challenge is due to the fact that many HCPs and reps have different medical knowledge, experience, expertise, and backgrounds, and the product bags of the HCPs and reps can vary. In order for a rep to be able to communicate effectively with a HCP, their medical knowledge and product bag should be commensurate with each other, such that the HCP has a possibility or use/need for prescribing the drug to patients that the rep is promoting. Another part of the challenge stems from the capacity and availability of reps, which can often vary depending on the rep schedules, locations, and other suggestions assigned or given to the reps. Moreover, although different teams within a pharma company generally target different HCP groups, there could sometimes be overlaps between the targeted groups.

The rep assignment model can be configured to perform one or more of the following steps. First, a master list of HCP-rep map or mapping can be created based on a “RepAccountAssigment” table in a database. The “RepAccountAssigment” table may be provided by a client or customer (e.g. a pharma company). The table may comprise a list of reps that are assigned to different HCPs. The list may include constraints or limits as to which reps can be assigned or matched for each HCP. The list can be generated by adding HCP-rep pairs which have at least one visit interaction in a specific timeframe (e.g. last six months) from ADLx. In some instances, a rep may change or switch teams over time. The team information for a rep can be updated based on the team changes by checking the latest updated rep team assignment in a CRM system, for example on a daily basis.

Next, the reps and HCPs who are not available for interaction on a certain day or timing are filtered out from the master list. The filtering can be based on “RepUnavailablePeriod” and “HCPUnavailablePeriod” tables. In some embodiments, the filtering can be based on a “RepAccountHold” table which looks at ineligible HCP-rep pairs. This table may contain a list of Rep-HCP pairs for which a rep had asked to suppress suggestions for next “N” number days. For example, dismissal or suppressing of suggestions with a reason being that “HCP is not available for the next 30 days.”

Next, the eligible products are added to the filtered master list. A HCP may be targeted for one or more (e.g. multiple) products. Similarly, a rep may be authorized to detail or promote one or more (e.g. multiple) products. Based on the “RepProductAuthorization” table and “AccountProduct” table, an intersecting list of eligible products for each HCP-Rep pair can be determined. Next, it is determined if there are any pairs with more than one product in the Rep-HCP table, which can vary by customer and product.

Next, the following factors are used for model score calculation for each HCP-rep pair. The factors may include (i) CRI (Customer Relationship Index), (ii) distance predicted by an anchor model, (iii) interaction count between the rep and HCP over a time period (e.g. last six months) and (iv) probability score generated by a Rep Engagement Model (REM). The above factors can be scaled, for example using a min-max scalar.

Next, the HCP-rep score for each pair is calculated by obtaining a mean of the scaled factors using the following:

Score(hᵢ,rⱼ)=mean(CRI(hᵢ,rⱼ),distance(hᵢ,rⱼ),REM . . . prob(hᵢ,rⱼ),interaction . . . count(hᵢ,rⱼ)

The score provides a degree of match between the rep and the HCP. The CRI (customer relationship index) model is described elsewhere herein, as well as PCT/US2021/019469 (titled “Systems and Methods for Enhancing Suggestions with an Effective Index”), which is incorporated herein by reference in its entirety. The CRI provides a rating of the strength of the relationship between a brand and the HCP. The distance estimate is based on a Rep Location Model (physical location and distance proximities of reps to HCPs). REM refers to the rep engagement ML model as described elsewhere herein, which outputs a rep engagement probability (i.e. probability that the rep will engage with a HCP based on a certain suggestion). The components are normalized. For example, the model score is a weighted average of the components. The weights can be optimized based on historical data, whereby rep engagement with HCP is an objective for the optimization.

The scores can be applied to HCP-rep pairs where the product bag of a rep overlaps with the product bag of the HCP. If there is no overlap between the product bags of the rep and HCP, the score is set to zero. When a score is zero, the rep is not assignable to the HCP. Rep capacity can be calculated based on parameters assigned to each channel reflecting operation time required by contact type. In some embodiments, a customer (e.g. pharma company) can provide their own rep assignment model with a BYOM (bring your own model) API, provided the scoring is on the same scale and can be interpreted as required by the rep selection algorithm.

The final step is the rep selection step. The score described above can be used for selection of reps for each suggestion or suggested action. Suggestions post optimization can be sorted by value from high to low based on the above calculated score. Reps can be assigned in order of suggestion value, assigning reps to higher value suggestions before lower value suggestions. For each HCP, a rep can be selected from a list of ‘still available reps’ based on the score of the rep-HCP pair as obtained from the rep assignment model, from highest to lowest. As the reps are being assigned, the availability of the reps is updated. The process is repeated iteratively until all of the output suggestions (for rep-HCP pairs) are exhausted. If no rep is found for a suggestion or suggested action, the suggestion will not be published.

### Campaign Management and Journey

Next, campaign management in the context of journey initiation and management is described. Oftentimes, customers (e.g. pharma companies and their marketing managers) will create a plan for reps to contact HCPs in certain ways. The plan may comprise a list of candidate HCPs from which to select a set of HCPs, for certain days for specific actions. Those campaigns or journeys typically have a start date, and sometimes they may have an end date as well. An example of a campaign with end date may be a conference invitation. For example, a conference may be planned for a certain date. As the date approaches, the reps are reminded to send messages to invite certain HCPs to the conference. There may be certain goals, for example, to achieve at least a certain number of HCPs to sign up for and turn up at the conference.

A journey is a series of steps in an attempt to better engage or communicate with a target audience. A journey may have any target audience (e.g. different groups or segments of HCPs). A journey may have a broad segment audience. Actions and specific options may be associated with each journey. A journey may have content of interest to the target audience.

Goals can be defined for each campaign. An example of a goal may be having at least a certain number of HCPs to sign up for a webinar. Requirements or constraints may be set, for example to accomplish the goal through at least a certain number of in-person rep visits to HCPs.

Journey initiation may include first (1) defining the different use cases. (2) processing the steps. (3) perform action evaluation, and (4) perform optimization. The campaign tracking and key results can be integrated with omni-channel (OC) business intelligence (BI). Each of the above is described in detail next.

### (1) Use Cases

In an example, two different use cases may be defined. Each use case may have a broad segment audience definition based on tier (e.g. HCP segment/tier), content of interest to target the desired audience, a duration (e.g. a begin date, or begin and end dates), and response targets (what is the overall response to be elicited from the audience, for each option as described below). As an example, a first use case may be an email campaign by a customer (e.g. pharma company) to promote HCP appreciation of a product (e.g. drug) efficacy. The campaign may include a plurality of options for actions taken. Each option may comprise a single action or a sequence of actions. For example, a first option may be a single email sent by the marketing group of the customer to HCPs. A second option may be a sequence of emails (e.g. two emails) sent by the marketing group of the customer to HCPs. A third option may be an email sent by the marketing group of the customer to HCPs, followed by an email from reps to the HCPs. The emails can relate to product efficacy as well as patient management. Content affinity can be used to differentiate HCP response probabilities between product efficacy and patient management, by changing the sequence of actions in any of the above options.

As another example, a second use case may be a webinar campaign, that may relate to a conference event invitation to the HCPs. Similarly, the webinar campaign may include a plurality of options for actions taken. Each option may comprise a single action or a sequence of actions. For example, a first option may be a single email sent by the marketing group of the customer to HCPs. A second option may be a single email sent by reps to HCPs. A third option may be a single in-person visit by the reps to the HCPs.

### (2) Processing

After the use cases have been defined, an action candidate processing (ACP) step is used to process journey definitions and create action-suggestion candidates. The data infrastructure of these action-suggestion candidates is in a format that is capable of being handled and further processed by the CIE. As an example, the action-suggestion candidates can be in a JSON package/Parquet. A consolidation/normalization step may be performed on these action-suggestion candidates as described elsewhere herein.

Next, the CIE performs pre-processing on the action-suggestion candidates, by identifying from the JSON package that this is a journey initiation and subsequently a plurality of journey initiation suggestions. Next, an initial campaign optimization is run to generate a campaign plan. Next, the campaign plan is transformed into actions (e.g. daily actions) for the reps. In some embodiments, re-optimization may be performed on a regular basis (e.g. the models herein can be updated daily or more often). Next, the tracking results are provided as feedback back to the system.

As an example of the optimization and re-optimization process. A response model may determine that a HCP has a first probability of responding to a specific type of email from a rep, and a second probability of being willing to engage with the rep in an in-person visit. The response model can be updated as the campaign progresses. Depending on the length of the campaign (days, weeks, or months), the campaign plan can be updated on a regular basis (e.g. daily or more often) depending on the updates to the response model. Re-optimization can occur using new updated probability assessments from the response model. Accordingly, using the above, each time a user sets up a campaign, the results can be automatically tracked, compiled and fed back to the system to automatically modify/update the models.

### (3) Action Evaluation

In some cases, when a journey is multi-stage, the options can be evaluated using decision analysis tree. An action value function incorporates success probabilities and impact evaluation including action costs, as well as any other considerations that are relevant. Constrained optimization is performed using an overall value maximization objective, to determine the value of each option.

### (4) Optimization

Optimization includes evaluating each option to select the optimal option, which can be solved for using segmented constrained linear programming. The campaign plan can be optimized/re-optimized on a frequent (e.g. daily) basis as described elsewhere herein.

Any of the data infrastructure systems herein may include one or more server computer systems for physically storing and processing data. The server computer systems may be housed at one or more facilities. If data storage is decentralized, the data infrastructure system may use a cloud storage model. Data stored within the data infrastructure system may be managed by one or more types of database (e.g., a relational database or a non-relational database), using languages such as SQL or NoSQL. Some of the databases in the database may be implemented using various standard data structures, such as an array, hash, (linked) list, struct, structured text file (e.g., XML), table, JavaScript™ Object Notation (JSON), NOSQL and/or the like. Such data structures may be stored in memory and/or in (structured) files. The database may comprise object-oriented databases. Object databases can include a number of object collections that are grouped and/or linked together by common attributes: they may be related to other object collections by some common attributes. Object-oriented databases may perform similarly to relational databases with the exception that objects are not just pieces of data but may have other types of functionality encapsulated within a given object. The database may include a graph database that uses graph structures for semantic queries with nodes, edges and properties to represent and store data. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated. The data infrastructure system may also include networking equipment for providing data to various parts of the system, including to end users. The networking equipment may provide access to the Internet or to a local network (e.g., a local are network (LAN)), or to a wide area network (WAN). The networking equipment may include routers, switches, cabling, access points, gateways, and other networking hardware. The data infrastructure system may also include software to authenticate and authorize users for access to various parts of the system. The data infrastructure system may also include software to pre-process data (e.g., to compress or deduplicate the data).

Any of the data analytics systems herein may process the data to generate analytics and predictions useful for increasing effectiveness of communications between pharma reps and health care providers (HCPs). The data analytics system may include computer hardware and software for generating machine-learned predictions regarding communication methods, and additional hardware and software to convert these predictions into scores or probabilities which convey information to pharma reps about best communication practices. The data analytics system may interface with various portions of the data infrastructure system. For example, data infrastructure components may send intermediate or final results to various portions of the data analytics system. The data analytics system may access databases within the data infrastructure system to extract data used for machine learning processes. The data analytics system may implement one or more machine learning pipelines to train machine learning models, automate machine learning tasks, provide results to various components of the system, and perform other data processing tasks. A machine learning pipeline may include a registry with one or more machine learning models to be implemented by the system.

Any of the customer relationship management systems herein may provide or generate electronic reports to pharma reps or their management, to show various metrics with respect to targeting HCPs, automate sales and marketing processes, and provide additional data to such users and other stakeholders within a user interface. The user interface may be a graphical user interface. The CRM system may receive recommended actions for optimizing performance with respect to long-term and short-term goals and present them to one or more users.

Any of the machine learning models may receive pre-processed data or may pre-process data. For example, a machine learning model may alter the dimensionality of the input data. The machine learning model may create an encoding or a representation of the input data. The machine learning models may be conducted over multiple epochs or iterations. In some embodiments, the machine learning model may reserve a validation set of data to validate the training of the algorithm, to determine whether more training is necessary. The trained model may then be tested on data to generate the predictions.

The machine learning model may use supervised machine learning (ML) algorithms. A supervised ML algorithm can be trained using labeled training inputs, i.e., training inputs with known outputs. The training inputs can be provided to an untrained or partially trained version of the ML algorithm to generate a predicted output. The predicted output can be compared to the known output, and if there is a difference, the parameters of the ML algorithm can be updated. A semi-supervised ML algorithm can be trained using a large number of unlabeled training inputs and a small number of labeled training inputs.

Any of the algorithms described herein may be neural networks. Neural networks may employ multiple layers of operations to predict one or more outputs. Neural networks can include one or more hidden layers situated between an input layer and an output layer. The output of each layer can be used as input to another layer, e.g., the next hidden layer or the output layer. Each layer of a neural network can specify one or more transformation operations to be performed on input to the layer. Such transformation operations may be referred to as neurons. The output of a particular neuron can be a weighted sum of the inputs to the neuron, adjusted with a bias and multiplied by an activation function, e.g., a rectified linear unit (ReLU) or a sigmoid function.

Training a neural network can involve providing inputs to the untrained neural network to generate predicted outputs, comparing the predicted outputs to expected outputs, and updating the algorithm's weights and biases to account for the difference between the predicted outputs and the expected outputs. Specifically, a cost function can be used to calculate a difference between the predicted outputs and the expected outputs. By computing the derivative of the cost function with respect to the weights and biases of the network, the weights and biases can be iteratively adjusted over multiple cycles to minimize the cost function. Training may be complete when the predicted outputs satisfy a convergence condition, e.g., a small magnitude of calculated cost as determined by the cost function.

As described herein, an optimization module can balance the plurality of machine learning outputs to provide an optimum output per HCP that balances a plurality of long term and short term goals. The optimization module can balance these goals by producing an output based on a weighing of several considerations. The output may or may not apply weights to a plurality of quantitative values associated with machine learned predictions relating to sales or communication effectiveness between reps and HCPs, before calculating an average, sum, or product of the quantitative values. The optimization module may have weight categories associated with such predictions, such as urgency, value, content affinity, channel affinity, feasibility, and cost. Following the weighting, the optimization module may implement a set of constraints. The short term goals maximized by the optimization module may include assisting with access challenges, reacting to HCP-led engagement, sharing new clinical data, or coordinating around an event. The long term goals optimized for may include targeting and segmentation of health care providers and providing a more engaging customer experience. The optimization module may also be used to rank HCPs by priority to interact, balancing short term engagement needs with long term commercial opportunities.

The optimization module may optimize using an objective function. The objective function may be a product of various terms, including a perceived value of an HCP, an impact of communicating via a particular channel, a probability of a successful sale, a target sales value, a recency of a prior communication, and a frequency of communication. Implementing the objective function may produce a score relating to a value for a particular action taken by a pharma rep to an HCP. Following producing such scores, the optimization module may place constraints restricting actions that may be taken. Constraints may include restrictions on types of actions, types of communication channels time constraints on pharma reps, constraints on contact frequencies, or other constraints.

Whenever the term “at least.” “greater than.” or “greater than or equal to” precedes the first numerical value in a series of two or more numerical values, the term “at least.” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Computer Systems

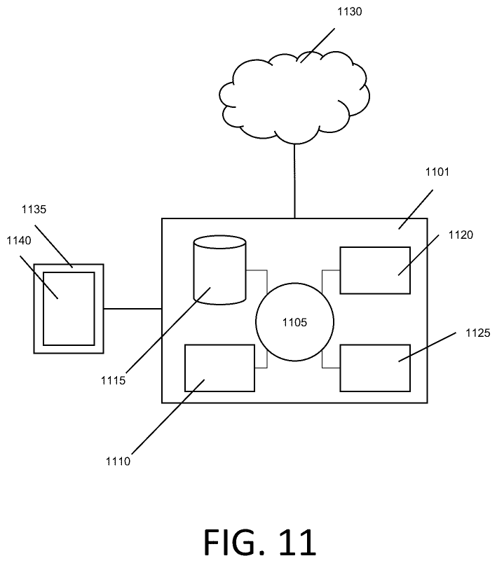

The present disclosure provides computer systems that are programmed to implement methods of the disclosure. FIG. 11 shows a computer system 1101 that is programmed or otherwise configured to machine learning model predictions subject to rules/constraints to optimize long-term and short-term goals. The computer system 1101 can regulate an regulate various aspects of producing suggestions of the present disclosure, such as, for example, performing machine learning analysis. The computer system 1101 can be an electronic device of a user or a computer system that is remotely located with respect to the electronic device. The electronic device of the user can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., a mobile user device). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables: copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, suggestions to pharma reps re communications to HCPs and also content. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, calculate the optimal engagement strategy across multiple channels between reps and HCPs.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-53. (canceled)

54. A method comprising:
receiving input data associated with promotion of a product to one or more target entities;
defining a plurality of temporal goals for attainment based at least in part on the input data, wherein the plurality of temporal goals is classified into at least two categories of different timescales;
providing the plurality of temporal goals and a set of constraints to an optimization model comprising an objective function, wherein the set of constraints comprise action-type constraints, channel constraints, channel capacity constraints, pacing constraints, and/or channel fatigue constraints across a plurality of channels, wherein the plurality of channels is used to enable or facilitate communications between one or more actor types and the one or more target entities; and
using the optimization model to generate a set of multi-dimensional actions with predicted action values for at least a subset of the plurality of channels, wherein the predicted action values collectively maximize an impact value of the objective function while balancing a feasibility of the plurality of temporal goals with respect to one another.

55. The method of claim 54, wherein the set of multi-dimensional actions with predicted action values are generated based on one or more of the following: a value of the one or more target entities, a relative impact value of the actions, a probability of success of the actions, or a timing value of the actions.

56. The method of claim 54, wherein the one or more actor types comprise one or more enumerated actors including sales representatives, district managers, or medical science liaisons that are associated with the promotion of the product to the one or more target entities.

57. The method of claim 56, wherein the one or more actor types comprise automated systems including marketing automation systems or web portal management systems.

58. The method of claim 55, wherein the product comprises one or more drug products or medical devices, and wherein the one or more target entities comprise one or more healthcare providers (HCPs), healthcare organizations (HCOs), or healthcare institutional accounts.

59. The method of claim 54, wherein the input data comprises market data, strategies, analytics, context, campaigns or models from one or more sources.

60. The method of claim 59, wherein the one or more sources comprise databases for customer relationship management (CRM), marketing automation systems, website target management systems, customer data platforms (CDP), market segmentation, sales, a pharmaceutical company's proprietary databases, and/or third party provided/generated data.

61. The method of claim 54, wherein the plurality of temporal goals comprises at least one long-term goal on a first time scale ranging from about six to twelve months, and a plurality of short-term goals on a second timescale that is daily, weekly, or monthly.

62. The method of claim 54, wherein the plurality of temporal goals is expressed in a form of actions and/or responses to actions on the different timescales.

63. The method of claim 54, wherein the objective function is configured to return a value function that is a product of a targeted entity value, action impact values, engagement probabilities, success probabilities for achieving the plurality of goals, and time values of the actions.

64. The method of claim 57, wherein the set of multi-dimensional actions are designed to complement each other so as to enable cross-channel optimization across the subset of channels based at least in part on an execution capacity of the enumerated actors, capacity limits set of the automated systems, utilization or consumption of content, or usefulness/relevance of the content.

65. The method of claim 54, wherein the optimization model is configured to iteratively search for actions to maximize expected action values by applying and varying the plurality of constraints and parameters to (1) influence the predicted action values and (2) monitor changes in the predicted action values.

66. The method of claim 65, wherein the plurality of parameters comprises one or more of the following: cost, impact, urgency, recency, channel propensity, physical proximity, representative (rep) engagement, time to engage, account priority, or factor priority.

67. The method of claim 65, wherein the optimization model is configured to search through a range of predicted action values for the plurality of channels when the plurality of parameters is being varied subject to the set of constraints, in order to identify a subset of predicted action values that forms a basis for the set of multi-dimensional actions.

68. The method of claim 54, further comprising generating a campaign comprising the multi-dimensional actions, wherein the multi-dimensional actions are ordered along a timescale of the campaign.

69. The method of claim 68, wherein the timescale comprises a start date and an end date for performing the multi-dimensional actions of the campaign.

70. The method of claim 68, further comprising providing the campaign to an actor of the one or more actor types, wherein the actor is prompted to perform the multi-dimensional actions of the campaign.

71. The method of claim 70, further comprising, responsive to the prompting, performing at least one action of the multi-dimensional actions of the campaign to accomplish at least one temporal goal of the plurality of temporal goals.

72. The method of claim 71, further comprising determining a progress of the actor in accomplishing the at least one temporal goal of the plurality of temporal goals.

73. The method of claim 72, further comprising analyzing the progress of the actor for integration into omni-channel business intelligence.
